(19) United States
(12) Patent Application Publication
(10) Pub. No.: US 2025/0029732 A1
(43) Pub. Date: Jan. 23, 2025

(54) METHOD AND SYSTEM FOR VIRTUAL SURGICAL PROCEDURE

(71) Applicant: BIOTRONIK AG, Buelach (CH)

(72) Inventor: Azadeh Mehrabi, Meilen (CH)

(21) Appl. No.: 18/714,823

(22) PCT Filed: Nov. 30, 2022
(86) PCT No.: PCT/EP2022/083782

§ 371 Date: May 30, 2024

(30) Foreign Application Priority Data
Dec. 22, 2021 (EP) 21216806.6

(51) Int. Cl. G16H 50/50; A61B 34/10; G16H 30/40

(52) U.S. Cl. CPC G16H 50/50; A61B 34/10; G16H 30/40; A61B 2034/102; A61B 2034/105

(57) ABSTRACT

A computer-implemented method and system for virtual vascular intervention applies a machine learning algorithm (A1) to a pre-acquired first data set (DS1) of medical image data of a patient (P) and a second data set (DS2) of patient medical parameters and/or natural language data related to a medical condition of the patient (P) to identify at least one pathological feature. The identified at least one pathological feature is indicated in a three-dimensional virtual model (M) of at least one portion of the body of the patient (P). A virtual vascular intervention is executed using the generated three-dimensional virtual model (M) and at least one surgical tool. Haptic feedback (F) is provided to a user using operational data of the virtual vascular intervention.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2022/083782, which was filed Nov. 30, 2022, which application claimed priority from EP application Ser. No. 21/216,806.6, which was filed Dec. 22, 2021.

### FIELD OF THE INVENTION

The invention relates to a computer implemented method for a virtual surgical procedure, virtual surgical procedure, in particular for vascular interventions, which may be used for virtual surgical procedure.

### BACKGROUND

The current utilization of advanced surgery preparation and aiding systems such as Image Guided and Navigation Systems which are based on pre-registered 3D imageries, are limited in assisting the surgeons. Also, surgeries are time-sensitive, for example, due to various procedures including temporary vessel clamping in specific areas. Therefore, the efficiency of the procedure is highly critical and detailed planning based on the patient specific local geometry and physical properties are fundamental. To achieve a new level of pre-surgery preparation, 3D CT and MRI images are being increasingly utilized. However, those images offer only minor benefits, standing alone, for surgery rehearsal.

### SUMMARY OF THE INVENTION

Preferred embodiments provide an improved method for virtual surgical procedure, in particular for vascular interventions, giving the medical practitioner a realistic visual model of the surgical site. A preferred computer-implemented method for virtual vascular interventions includes providing (S1) a pre-acquired first data set (DS1) of medical image data of a patient (P). A pre-acquired second data set (DS2) of patient medical parameters and/or natural language data related to a medical condition of the patient (P) is provided. A three-dimensional virtual model (M) of at least a portion of the body of the patient (P) based on the pre-acquired first data set (DS1) and on the pre-acquired second data set (DS2) is generated. A machine learning algorithm (A1) is applied to the pre-acquired first data set (DS1) and the second data set (DS2) to identify at least one pathological feature includes the pre-acquired first data set (DS1) and the pre-acquired second data set (DS2). The at least one pathological feature is indicated in the three-dimensional virtual model (M). A virtual vascular intervention is executed using the generated three-dimensional virtual model (M) and at least one surgical tool. Haptic feedback (F) is provided to a user using operational data of the virtual vascular intervention. The operational data includes data of a position of the surgical tool within the three-dimensional virtual model (M) and/or data related to the at least one surgical tool.

### BRIEF DESCRIPTION OF THE DRAWINGS

For a more complete understanding of the present invention and advantages thereof, reference is now made to the following description taken in conjunction with the accompanying drawings. The invention is explained in more detail below using exemplary embodiments, which are specified in the schematic figures of the drawings, in which:

FIG. 1 shows a flowchart of a computer implemented method for virtual vascular intervention, in particular for vascular interventions, according to a preferred embodiment of the invention;

FIG. 2 shows a flowchart of a computer implemented method for providing a trained machine learning algorithm for identifying at least one pathological feature including a pre-acquired first data set of medical image data of a patient and a pre-acquired second data set of patient medical parameters and/or natural language data related to a medical condition of the patient, according to the preferred embodiment of the invention; and

FIG. 3 shows a schematic illustration of a system for virtual vascular intervention, in particular for vascular interventions, according to the preferred embodiment of the invention.

### DETAILED DESCRIPTION OF THE PREFERRED EMBODIMENTS

Furthermore, the invention relates to a computer-implemented method for providing a trained machine learning algorithm for identifying at least one pathological feature that includes a pre-acquired first data set of medical image data of a patient and a pre-acquired second data set of patient medical parameters and/or natural language data related to a medical condition of the patient.

In addition, the invention relates to a system for virtual surgical procedure, in particular for vascular interventions.

A computer-implemented method provides a trained machine learning algorithm for providing a trained machine learning algorithm for identifying at least one pathological feature comprised by a pre-acquired first data set of medical image data of a patient and a pre-acquired second data set of patient medical parameters and/or natural language data related to a medical condition of the patient.

The present invention provides a computer-implemented method for virtual vascular interventions.

Advantageously, the computer-implemented method for virtual vascular interventions may be used in the surgical training of a medical practitioner.

The method includes providing a pre-acquired first data set of medical image data of a patient and providing a pre-acquired second data set of patient medical parameters and/or natural language data related to a medical condition of the patient.

Furthermore, the method includes generating a three-dimensional virtual model of at least a portion of the body of the patient based on the pre-acquired first data set of medical image data of the patient and on the pre-acquired second data set of patient medical parameters and/or natural language data related to a medical condition of the patient.

In addition, the method includes applying a machine learning algorithm to the pre-acquired first data set of medical image data of a patient and the second data set of patient medical parameters and/or natural language data related to a medical condition of the patient to identify at least one pathological feature included by the pre-acquired first data set of the medical image data of the patient and the pre-acquired second data set of patient medical parameters and/or natural language data related to a medical condition of the patient, wherein the identified at least one pathological feature is indicated in the three-dimensional virtual model of the at least one portion of the body of the patient.

The method moreover includes executing a virtual vascular intervention using the generated three-dimensional virtual model and a surgical tool, and generating haptic feedback to a user using operational data of the virtual vascular intervention, wherein the operational data includes data of a position of the surgical tool within the three-dimensional virtual model and/or data related to the surgical tool. The haptic feedback may particularly give a feedback whether the surgical tool or device is used or placed correctly. In addition or alternatively, the haptic feedback may give the user a realistic life-like feeling.

The present invention further provides a computer implemented method for providing a trained machine learning algorithm for identifying at least one pathological feature included by a pre-acquired first data set of medical image data of a patient and a pre-acquired second data set of patient medical parameters and/or natural language data related to a medical condition of the patient.

The method includes providing a first training data set including a pre-acquired first data set of medical image data of a patient and providing a second training data set including an identified at least one pathological feature included by the pre-acquired first data set of the medical image data of the patient and the pre-acquired second data set of patient medical parameters and/or natural language data related to a medical condition of the patient.

Furthermore, the method includes training the machine learning algorithm by an optimization algorithm which calculates an extreme value of a loss function for identifying the at least one pathological feature included by the pre-acquired first data set of the medical image data of the patient and the pre-acquired second data set of patient medical parameters and/or natural language data related to a medical condition of the patient.

The present invention moreover provides a system for virtual vascular interventions. The system includes code and hardware for providing a pre-acquired first data set of medical image data of a patient and code and hardware for providing a pre-acquired second data set of patient medical parameters and/or natural language data related to a medical condition of the patient.

Furthermore, the system includes code and hardware for generating a three-dimensional virtual model of at least a portion of the body of the patient based on the pre-acquired first data set of medical image data of the patient and on the pre-acquired second data set of patient medical parameters and/or natural language data related to a medical condition of the patient.

In addition, the system includes code and hardware for applying a machine learning algorithm to the pre-acquired first data set of medical image data of a patient and the second data set of patient medical parameters and/or natural language data related to a medical condition of the patient to identify at least one pathological feature included by the pre-acquired first data set of the medical image data of the patient and the pre-acquired second data set of patient medical parameters and/or natural language data related to a medical condition of the patient, wherein the identified at least one pathological feature is indicated in the three-dimensional virtual model of the at least one portion of the body of the patient.

The system moreover includes code and hardware for executing a virtual vascular intervention using the generated three-dimensional virtual model and code and hardware for generating haptic feedback to a user using operational data of the virtual vascular intervention.

It is an idea of the present invention to provide surgeons with a rehearsal and preparation tool that provides them with a realistic visual model with physical tissue properties, such as, for example, the inner geometry of a vessel, an organ properties or a problematic site. Most importantly, it enables a “full immersion” surgical tool that encompasses: realistic “life-like” 3D display of the patient-specific area of surgery, modeling of the local patient-specific area of surgery geometry and physical properties, an interface enabling manipulation of the patient-specific area of the surgery model and to virtually perform surgical actions such as cutting, shifting and clamping and an interface to provide feedback cues to the surgeon. Also a vascular intervention may be simulated with different surgical tools or devices under different circumstances. The surgical tool may be a catheter or a catheter system. The surgical tool may be a virtual surgical tool.

Machine learning algorithms are based on using statistical techniques to train a data processing system to perform a specific task without being explicitly programmed to do so. The goal of machine learning is to construct algorithms that can learn from data and make predictions. These algorithms create mathematical models that can be used, for example, to classify data or to solve regression type problems.

Operational data of the virtual vascular intervention is considered to be e.g. data of a position of a surgical tool within a patient anatomical structure and/or data on which surgical tool is currently being used.

According to an aspect of the invention, based on a third data set including the pre-acquired first data set of medical image data of a patient, the pre-acquired second data set of patient medical parameters and/or natural language data related to a medical condition of the patient and/or the generated three-dimensional virtual model of at least a portion of the body of the patient and a fourth data set including the at least one identified pathological feature, the least one surgical tool and/or at least one implantable medical device is recommended for use in the virtual vascular intervention.

The surgeon can thus advantageously practice the surgery procedure in advance using multiple surgical tools and/or at least one implantable medical device in order to determine the best possible tool and/or medical device. In addition, the surgeon may vary the surgical tool or device in order to determine which one is better suited for him for the vascular intervention and/or which type of treatment fits the patient best. According to a further aspect of the invention, for the at least one surgical tool and/or the at least one implantable medical device recommended for use in the virtual vascular intervention, a usefulness probability is calculated based on medical data of further patients having a similar medical condition. The medical data of the patients having a similar medical condition can thus advantageously serve as further data points aiding the decision of the surgeon on which tool and/or medical device to choose for the present patient.

According to a further aspect of the invention, a further machine learning algorithm and/or a rule-based algorithm is applied to the third data set including the pre-acquired first data set of medical image data of a patient, the pre-acquired second data set of patient medical parameters and/or natural language data related to a medical condition of the patient and/or the generated three-dimensional virtual model of at least a portion of the body of the patient and the fourth data set including the at least one identified pathological feature for classifying at least one class representing at least one further patient having a closest matching health condition. By classifying the at least one further patient having a closest matching health condition, additional useful information on e.g. surgical tools and/or medical devices used can be obtained.

According to a further aspect of the invention, the at least one class representing the at least one further patient having a closest matching health condition is outputted by the further machine learning algorithm and/or a rule-based algorithm in an order of similarity to the third data set including the pre-acquired first data set of medical image data of a patient, the pre-acquired second data set of patient medical parameters and/or natural language data related to a medical condition of the patient and/or the generated three-dimensional virtual model of at least a portion of the body of the patient and the fourth data set including the at least one identified pathological feature. This advantageously provides the medical practitioner with a list of further information that can aid the medical practitioner in decision-making.

According to a further aspect of the invention, a position, size and/or severity of the pathological feature identified by the machine learning algorithm is annotated in the three-dimensional virtual model of the at least one portion of the body of the patient. This automatic annotation provides the medical practitioner with a convenient overview of the one more pathological feature in the three-dimensional virtual model of the patient.

According to a further aspect of the invention, the pre-acquired first data set of medical image data of a patient includes CT-data, MRI-data, angiographs, and/or ultrasound-data. Thus, advantageously multiple medical image data sources can be used and/or combined for generating the three-dimensional virtual model.

According to a further aspect of the invention, upon generation of the three-dimensional virtual model of at least a portion of the body of the patient, a medical practitioner information request is triggered requesting if the medical practitioner accepts or rejects the three-dimensional virtual model of at least a portion of the body of the patient and the identified at least one pathological feature included by the pre-acquired first data set of the medical image data of the patient and the pre-acquired second data set of patient medical parameters and/or natural language data related to a medical condition of the patient.

The medical practitioner can thus advantageously based on professional experience and/or knowledge of the specific patient condition of the present patient accept or reject the automatically generated three-dimensional virtual model.

According to a further aspect of the invention, if in response to the medical practitioner information request the medical practitioner rejects the three-dimensional virtual model of at least a portion of the body of the patient and the identified at least one pathological feature included by the pre-acquired first data set of the medical image data of the patient and the pre-acquired second data set of patient medical parameters and/or natural language data related to a medical condition of the patient, a further medical practitioner information request is triggered requesting to provide reasons for the rejection.

The information provided by the medical practitioner in submitting the further medical practitioner information request can thus advantageously be used to train and/or update the machine learning algorithm in order to improve accuracy of future predictions.

According to a further aspect of the invention, upon submitting the further medical practitioner information request, the three-dimensional virtual model of at least a portion of the body of the patient and/or the identified at least one pathological feature is corrected by editing predetermined features or is replaced by a replacement data set. The medical practitioner is thus able to edit or correct any pathological features deemed to be inaccurate.

According to a further aspect of the invention, the virtual vascular intervention using the generated three-dimensional virtual model is recorded, wherein a recording of the executed virtual vascular intervention is used to control a surgical robot to execute a vascular intervention. The pre-recorded vascular intervention can thus advantageously be used to conduct an actual vascular intervention at a later time.

According to a further aspect of the invention, the virtual vascular intervention using the generated three-dimensional virtual model is analyzed to determine feedback data including a duration of the virtual vascular intervention, a patient health condition after the virtual vascular intervention, a usage and/or a type of surgical tools and/or implantable medical devices and/or ID data of the medical practitioner performing the virtual vascular intervention. This data can advantageously be stored and used to predict said values for future vascular interventions having similar parameters. According to a further aspect of the invention, based on the feedback data is used to train the further machine learning algorithm and/or update rules of the rule-based algorithm. The further machine learning algorithm and/or the rule-based algorithm are thus continuously updated and/or trained to enhance their performance.

The herein described features of the computer implemented method for virtual vascular intervention, in particular for vascular interventions, are also disclosed for the system for virtual vascular intervention, in particular for vascular interventions and vice versa.

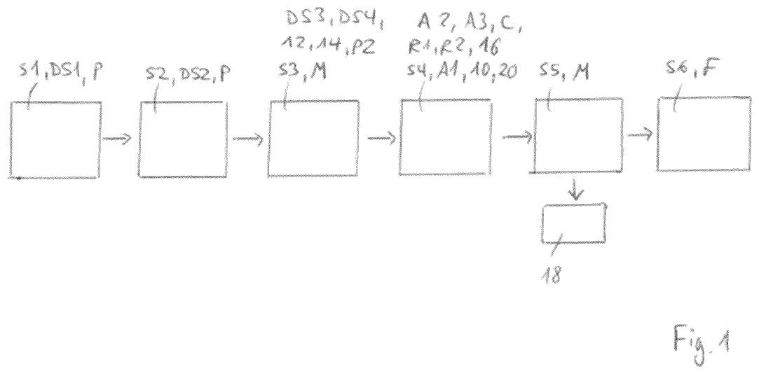

The computer implemented method of FIG. 1 for virtual surgical procedure, in particular for vascular interventions, includes providing S1 a pre-acquired first data set DS1 of medical image data of a patient P and providing S2 a pre-acquired second data set DS2 of patient medical parameters and/or natural language data related to a medical condition of the patient P.

The method further includes generating S3 a three-dimensional virtual model M of at least a portion of the body of the patient P based on the pre-acquired first data set DS1 of medical image data of the patient P and on the pre-acquired second data set DS2 of patient medical parameters and/or natural language data related to a medical condition of the patient P.

In addition, the method includes applying S4 a machine learning algorithm A1 to the pre-acquired first data set DS1 of medical image data of a patient P and the second data set DS2 of patient medical parameters and/or natural language data related to a medical condition of the patient P to identify at least one pathological feature 10 included by the pre-acquired first data set DS1 of the medical image data of the patient P and the pre-acquired second data set DS2 of patient medical parameters and/or natural language data related to a medical condition of the patient P, wherein the identified at least one pathological feature 10 is indicated in the three-dimensional virtual model M of the at least one portion of the body of the patient P.

The method moreover includes executing S5 a virtual surgical procedure using the generated three-dimensional virtual model M and generating S6 haptic feedback F to a user using operational data of the virtual surgical procedure.

Based on a third data set DS3 including the pre-acquired first data set DS1 of medical image data of a patient P, the pre-acquired second data set DS2 of patient medical parameters and/or natural language data related to a medical condition of the patient P and/or the generated three-dimensional virtual model M of at least a portion of the body of the patient P and a fourth data set DS4 including the at least one identified pathological feature 10, at least one surgical tool 12 and/or at least one implantable medical device 14 is recommended for use in the virtual surgical procedure.

For the at least one surgical tool 12 and/or the at least one implantable medical device 14 recommended for use in the virtual surgical procedure, a usefulness probability is calculated based on medical data of further patients P2 having a similar medical condition.

A further machine learning algorithm A2 and/or a rule-based algorithm A3 is applied to the third data set DS3 including the pre-acquired first data set DS1 of medical image data of a patient P, the pre-acquired second data set DS2 of patient medical parameters and/or natural language data related to a medical condition of the patient P and/or the generated three-dimensional virtual model M of at least a portion of the body of the patient P and the fourth data set DS4 including the at least one identified pathological feature 10 for classifying at least one class C representing at least one further patient P2 having a closest matching health condition.

The at least one class C representing the at least one further patient P2 having a closest matching health condition is outputted by the further machine learning algorithm A2 and/or a rule-based algorithm A3 in an order of similarity to the third data set DS3 including the pre-acquired first data set DS1 of medical image data of a patient P, the pre-acquired second data set DS2 of patient medical parameters and/or natural language data related to a medical condition of the patient P and/or the generated three-dimensional virtual model M of at least a portion of the body of the patient P and the fourth data set DS4 including the at least one identified pathological feature 10.

A position, size and/or severity of the pathological feature 10 identified by the machine learning algorithm A1 is further annotated in the three-dimensional virtual model M of the at least one portion of the body of the patient P. The pre-acquired first data set DS1 of medical image data of a patient P includes CT-data, MRI-data and/or ultrasound-data.

Upon generation of the three-dimensional virtual model M of at least a portion of the body of the patient P, a medical practitioner information request R1 is triggered requesting if the medical practitioner accepts or rejects the three-dimensional virtual model M of at least a portion of the body of the patient P and the identified at least one pathological feature 10 included by the pre-acquired first data set DS1 of the medical image data of the patient P and the pre-acquired second data set DS2 of patient medical parameters and/or natural language data related to a medical condition of the patient P.

If in response to the medical practitioner information request R1 the medical practitioner rejects the three-dimensional virtual model M of at least a portion of the body of the patient P and the identified at least one pathological feature 10 included by the pre-acquired first data set DS1 of the medical image data of the patient P and the pre-acquired second data set DS2 of patient medical parameters and/or natural language data related to a medical condition of the patient P, a further medical practitioner information request R2 is triggered requesting to provide reasons for the rejection.

Upon submitting the further medical practitioner information request R2, the three-dimensional virtual model M of at least a portion of the body of the patient P and/or the identified at least one pathological feature 10 is corrected by editing predetermined features or is replaced by a replacement data set 16. The virtual surgical procedure using the generated three-dimensional virtual model M is recorded, wherein a recording of the executed virtual surgical procedure is used to control a surgical robot 18 to execute a surgical procedure.

The virtual surgical procedure using the generated three-dimensional virtual model M is analyzed to determine feedback data 20 including a duration of the virtual surgical procedure, a patient P health condition after the virtual surgical procedure, a usage of surgical tools 12 and/or implantable medical devices 14 and/or ID data of the medical practitioner performing the virtual surgical procedure. Based on the feedback data 20 is used to train the further machine learning algorithm A2 and/or update rules of the rule-based algorithm A3.

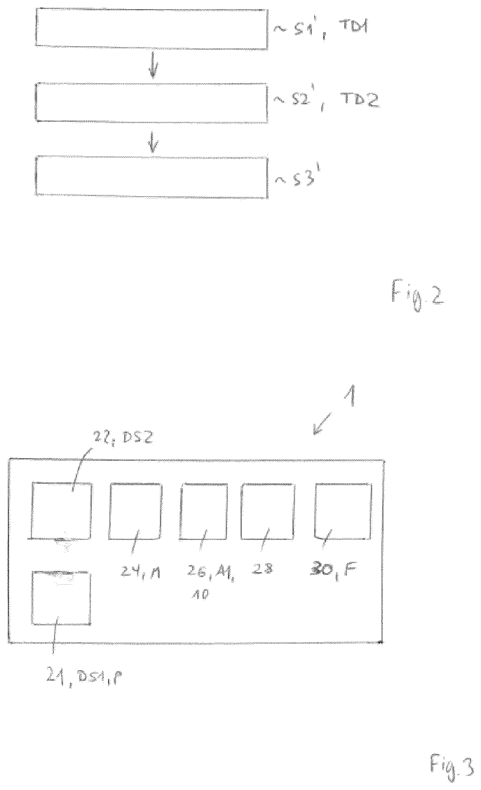

FIG. 2 shows a flowchart of a computer implemented method for providing a trained machine learning algorithm for identifying at least one pathological feature included by a pre-acquired first data set of medical image data of a patient and a pre-acquired second data set of patient medical parameters and/or natural language data related to a medical condition of the patient, according to the preferred embodiment of the invention.

The method includes providing S1′ a first training data set TD1 including a pre-acquired first data set DS1 of medical image data of a patient P and providing S2′ a second training data set TD2 including an identified at least one pathological feature 10 included by the pre-acquired first data set DS1 of the medical image data of the patient P and the pre-acquired second data set DS2 of patient medical parameters and/or natural language data related to a medical condition of the patient P.

In addition, the method includes training S3′ the machine learning algorithm A1 by an optimization algorithm which calculates an extreme value of a loss function for identifying the at least one pathological feature 10 included by the pre-acquired first data set DS1 of the medical image data of the patient P and the pre-acquired second data set DS2 of patient medical parameters and/or natural language data related to a medical condition of the patient P.

FIG. 3 shows a schematic illustration of a system for virtual surgical procedure, in particular for vascular interventions, according to the preferred embodiment of the invention.

The system includes code and hardware 21 for providing a pre-acquired first data set DS1 of medical image data of a patient P and code and hardware 22 for providing a pre-acquired second data set DS2 of patient medical parameters and/or natural language data related to a medical condition of the patient P.

Moreover, the system includes code and hardware 24 for generating a three-dimensional virtual model M of at least a portion of the body of the patient P based on the pre-acquired first data set DS1 of medical image data of the patient P and on the pre-acquired second data set DS2 of patient medical parameters and/or natural language data related to a medical condition of the patient P.

The system in addition includes code and hardware 26 for applying a machine learning algorithm A1 to the pre-acquired first data set DS1 of medical image data of a patient P and the second data set DS2 of patient medical parameters and/or natural language data related to a medical condition of the patient P to identify at least one pathological feature 10 included by the pre-acquired first data set DS1 of the medical image data of the patient P and the pre-acquired second data set DS2 of patient medical parameters and/or natural language data related to a medical condition of the patient P, wherein the identified at least one pathological feature 10 is indicated in the three-dimensional virtual model M of the at least one portion of the body of the patient P.

The system furthermore includes code and hardware 28 for executing a virtual surgical procedure using the generated three-dimensional virtual model M and code and hardware 30 for generating haptic feedback F to a user using operational data of the virtual surgical procedure.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

### REFERENCE SIGNS

1 system
10 pathological feature
12 surgical tool
14 implantable medical device
16 replacement data set
18 surgical robot
20 feedback data
21, 22, 24 code and hardware
26, 28, 30 code and hardware
A1 machine learning algorithm
A2 further machine learning algorithm
A3 rule-based algorithm
C class
DS1 first data set
DS2 second data set
DS3 third data set
DS4 fourth data set
F haptic feedback
M three-dimensional virtual model
P patient
P2 further patient(s)
R1 medical practitioner information request
R2 further medical practitioner information request
S1-S6 method steps
S1′-S3′ method steps
TD1 first training data set
TD2 second training data set

1. A computer-implemented method for virtual vascular interventions, comprising the steps of:
providing (S1) a pre-acquired first data set (DS1) of medical image data of a patient (P);
providing (S2) a pre-acquired second data set (DS2) of patient medical parameters and/or natural language data related to a medical condition of the patient (P);
generating (S3) a three-dimensional virtual model (M) of at least a portion of the body of the patient (P) based on the pre-acquired first data set (DS1) and on the pre-acquired second data set (DS2);
applying (S4) a machine learning algorithm (A1) to the pre-acquired first data set (DS1) and the second data set (DS2) to identify at least one pathological feature comprised by the pre-acquired first data set (DS1) and the pre-acquired second data set (DS2), wherein the at least one pathological feature is indicated in the three-dimensional virtual model (M);
executing (S5) a virtual vascular intervention using the generated three-dimensional virtual model (M) and at least one surgical tool; and
generating (S6) haptic feedback (F) to a user using operational data of the virtual vascular intervention, wherein the operational data comprises data of a position of the surgical tool within the three-dimensional virtual model (M) and/or data related to the at least one surgical tool.

2. The computer-implemented method of claim 1, comprising recommending at least one implantable medical device and/or the at least one surgical tool for use in the virtual vascular intervention based on a third data set (DS3) comprising the pre-acquired first data set (DS1), the pre-acquired second data set (DS2), and/or the generated three-dimensional virtual model (M) and a fourth data set (DS4) comprising the at least one identified pathological feature.

3. The computer-implemented method of claim 2, comprising calculating a usefulness probability based on medical data of other patients (P2) having a similar medical condition for the at least one surgical tool and/or the at least one implantable medical device.

4. The computer-implemented method of claim 3, wherein an additional machine learning algorithm (A2) and/or a rule-based algorithm (A3) is applied to the third data set (DS3), the pre-acquired second data set (DS2) and/or the generated three-dimensional virtual model (M), and the fourth data set (DS4) to classify at least one class (C) representing at least one additional patient (P2) having a closest matching health condition.

5. The computer-implemented method of claim 4, wherein the at least one class (C) is outputted by the additional machine learning algorithm (A2) and/or the rule-based algorithm (A3) in an order of similarity to the third data set (DS3), the pre-acquired second data set (DS2), and/or the generated three-dimensional virtual model (M) and the fourth data set (DS4).

6. The computer-implemented method of claim 1, wherein a position, size and/or severity of the at least one pathological feature is annotated by the machine learning algorithm (A1) in the three-dimensional virtual model (M).

7. The computer-implemented method of claim 1, wherein the pre-acquired first data set (DS1) comprises CT-data, MRI-data and/or ultrasound-data.

8. The computer-implemented method of claim 1, wherein upon generation of the three-dimensional virtual model (M) a medical practitioner information request (R1) is triggered requesting if the medical practitioner accepts or rejects the three-dimensional virtual model (M) and the at least one pathological feature.

9. The computer-implemented method of claim 8, wherein, if in response to the medical practitioner information request (R1) the medical practitioner rejects the three-dimensional virtual model (M) and/or the at least one pathological feature, an additional medical practitioner information request (R2) is triggered requesting reasons of the medical practitioner.

10. The computer-implemented method of claim 9, wherein upon submitting the additional medical practitioner information request (R2), the three-dimensional virtual model (M) and/or the identified at least one pathological feature is corrected by editing predetermined features or is replaced by a replacement data set.

11. The computer-implemented method of claim 1, comprising recording the virtual vascular intervention and using a recording of the virtual vascular intervention to control a surgical robot to execute a vascular intervention.

12. The computer-implemented method of claim 4, wherein the virtual vascular intervention is analyzed to determine feedback data comprising a duration of the virtual vascular intervention, a patient health condition after the virtual vascular intervention, a usage of the at least on surgical tool and/or at least one implantable medical device, and/or ID data of a medical practitioner performing the virtual vascular intervention.

13. The computer-implemented method of claim 12, comprising training the additional machine learning algorithm (A2) and/or updating rules of the rule-based algorithm (A3) based upon the feedback data.

14. A computer-implemented method for providing a trained machine learning algorithm (A1) for identifying at least one pathological feature related to a medical condition of the patient (P), comprising the steps of:
providing (S1′) a first training data set (TD1) comprising a pre-acquired first data set (DS1) of medical image data of a patient (P);
providing (S2′) a second training data set (TD2) comprising an identified at least one pathological feature comprised by the pre-acquired first data set (DS1) of the medical image data of the patient (P) and a pre-acquired second data set (DS2) of patient medical parameters and/or natural language data related to a medical condition of the patient (P); and
training (S3′) the machine learning algorithm (A1) by an optimization algorithm which calculates an extreme value of a loss function for identifying the at least one pathological feature.

15. A system for virtual vascular intervention, comprising:
means for providing a pre-acquired first data set (DS1) of medical image data of a patient (P);
means for providing a pre-acquired second data set (DS2) of patient medical parameters and/or natural language data related to a medical condition of the patient (P);
means for generating a three-dimensional virtual model (M) of at least a portion of the body of the patient (P) based on the pre-acquired first data set (DS1) and on the pre-acquired second data set (DS2);
means for applying a machine learning algorithm (A1) to the pre-acquired first data set (DS1) and the second data set (DS2) to identify at least one pathological feature comprised by the pre-acquired first data set (DS1) and the pre-acquired second data set (DS2), wherein the identified at least one pathological feature is indicated in the three-dimensional virtual model (M);
means for executing a virtual vascular intervention using the generated three-dimensional virtual model (M) and a virtual surgical tool; and
means for generating haptic feedback (F) to a user using operational data of the virtual vascular intervention, wherein the operational data comprises data of a position of the surgical tool within the three-dimensional virtual model (M) and/or data related to the surgical tool.
